(19) United States
(12) Patent Application Publication
(10) Pub. No.: US 2010/0004601 A1
(43) Pub. Date: Jan. 7, 2010

(54) MALECOT GASTRONOMY TUBE WITH SPLITTABLE SHEATH

(75) Inventor: Michael D. Deckard, Solsberry, IN (US)

Correspondence Address: BRINKS HOFER GILSON & LIONE, P.O. BOX 10395, CHICAGO, IL, 60610, US

(73) Assignee: Cook Incorporated, Bloomington, IN (US)

(21) Appl. No.: 12/167,763

(22) Filed: Jul. 3, 2008

(51) Int. Cl. A61M 39/02

(52) U.S. Cl. 604/171

(57) ABSTRACT

A gastronomy tube device includes an expandable distal retention portion that is self-expanding. The device includes a removable sheath that circumferentially expands the self-expanding distal retention portion. The distal retention portion preferably is biased into an outer diameter that is greater when released from the sheath than when constrained by it.

### TECHNICAL FIELD

The present patent application relates generally to medical devices, particularly to a gastronomy tube device configured with an expandable member and a sheath configured to constrain the expandable member during deployment.

### BACKGROUND

Gastronomy tubes, also known as gastric feeding tubes, gastro-tubes, or “g-tubes” are well-known in the art. As shown in FIG. 1C, a gastronomy tube 150 may be placed through a stoma 152 in the abdominal wall 154 of a patient to provide direct access to the patient's stomach 162 for gastric decompression or drainage, or for delivery of medication or nutrition. The stoma 154 generally is formed by an incision that heals to form a patent passage. The gastronomy tube 150 typically is retained in the stomach 162 by an expandable member such as a balloon 156 that is deployed/expanded in the stomach.

Malecots are also well-known within the medical art, including as expandable retention members for gastronomy tubes. In particular, they are known for use in catheter retention applications and other applications where it is desirable to have an expandable member to hold a tubular device within a patient body. For example, malecots are known for use with drainage tubes and feeding tubes. As shown in FIG. 1A, an existing malecot design 100 typically is introduced in an undeployed near-cylindrical configuration of an outer sheath 102 affixed to an introducer device 104. As shown in FIG. 1B, the expandable malecot wing portion 106 of the outer sheath 102 is deployed by moving the outer sheath 102 distally relative to the introducer device 104 and/or by moving the introducer device 104 proximally relative to the outer sheath 102. It may be advantageous to provide a gastronomy tube with a self-deploying expandable member.

### BRIEF SUMMARY

In one aspect, an embodiment of the present invention may include a medical device comprising a gastronomy tube configured as an elongate generally tubular body including a proximal end and a distal end with an elongate lumen disposed through its length, where the body includes a distal expandable region that is biased toward an expanded configuration, and a removable sheath disposed around and substantially constraining the expandable region in a non-expanded configuration having an outer diameter less than the expanded configuration.

In another aspect, an embodiment of the present invention may include a medical device comprising a gastronomy tube device configured as an elongate generally tubular body including a proximal end and a distal end with an elongate lumen disposed through substantially its entire length, where the body includes a distal self-expanding portion, and a removable sheath disposed around and substantially constraining the distal self-expanding portion in a non-expanded configuration having an outer diameter that is less than the expanded configuration.

And, in another aspect, an embodiment of the present invention may include a method of using a gastronomy tube device comprising the steps of: providing a gastronomy tube device comprising a self-expanding distal retention member that is constrained by a removable sheath; directing the distal retention member to a target site; and removing the sheath, allowing expansion of an outer diameter of the distal self-expanding retention member.

### BRIEF DESCRIPTION OF THE DRAWINGS

FIGS. 1A-1B show a malecot device;

FIG. 1C shows a gastric tube device installed in a diagrammatic patient;

FIG. 2A shows an assembled embodiment of a sheathed self-expanding gastronomy tube device;

FIG. 2B shows a disassembled sheath for a gastronomy tube device;

FIG. 2C shows a self-expanding body portion of the gastronomy tube device of FIG. 2A, illustrated as if circumferentially constrained;

FIG. 2D shows a deployed body portion of the gastronomy tube device of FIG. 2A;

FIGS. 3A-3C illustrate a method of installing the gastronomy tube device of FIG. 2A;

FIG. 3D illustrates a method of removing the gastronomy tube device of FIG. 2A;

FIG. 4A shows a different assembled embodiment of a sheathed self-expanding gastronomy tube device;

FIG. 4B shows a self-expanding body portion of the gastronomy tube device of FIG. 4A, illustrated as if circumferentially constrained; and

FIG. 4C shows a deployed body portion of the gastronomy tube device of FIG. 4A.

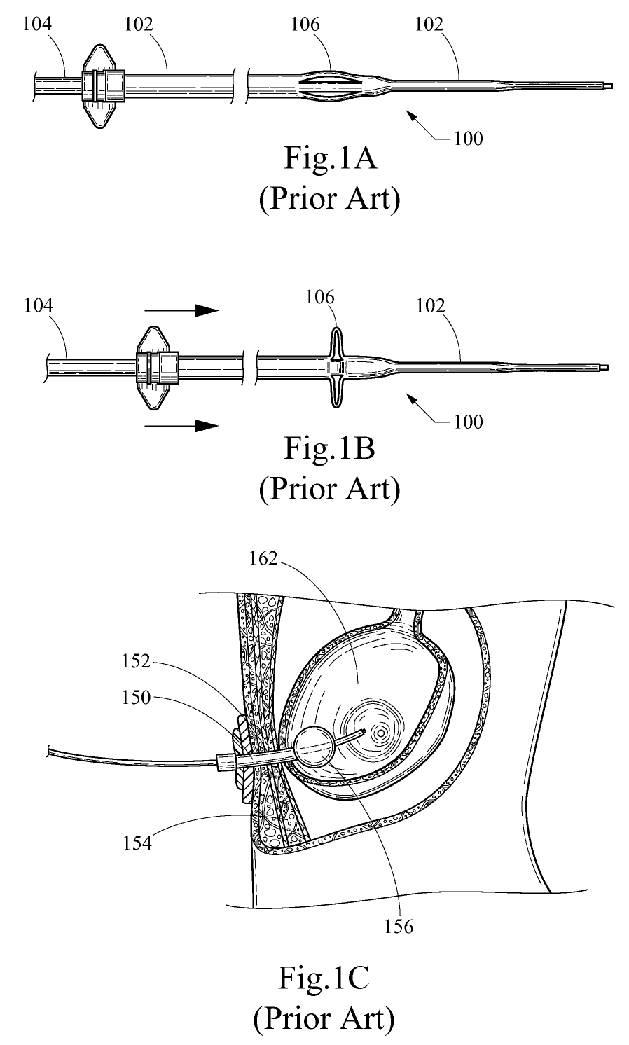

### DETAILED DESCRIPTION OF THE DRAWINGS AND VARIOUS EMBODIMENTS

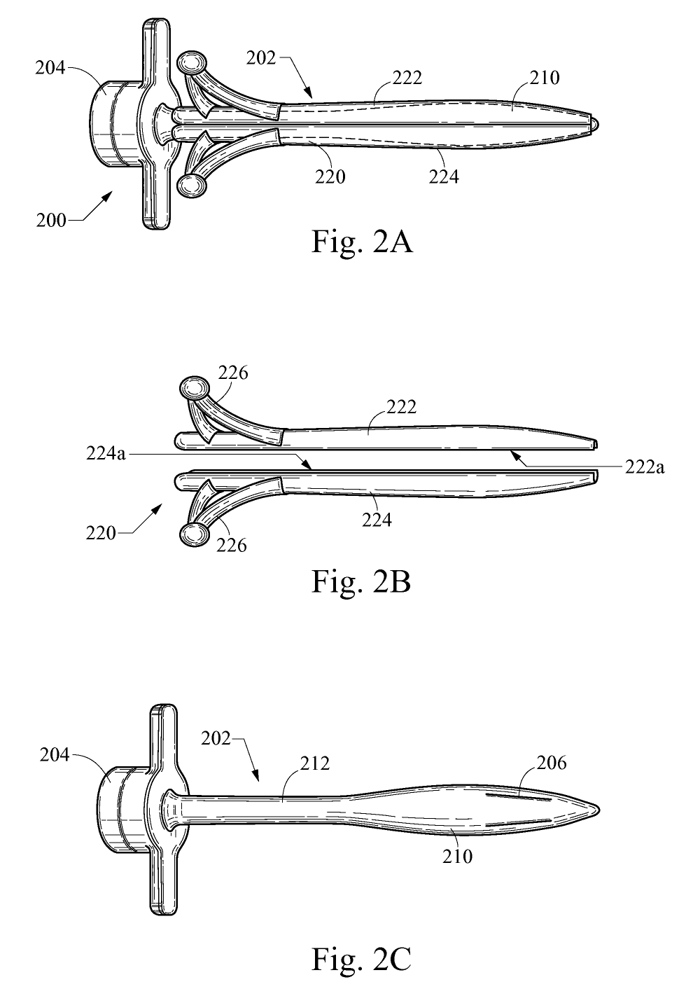

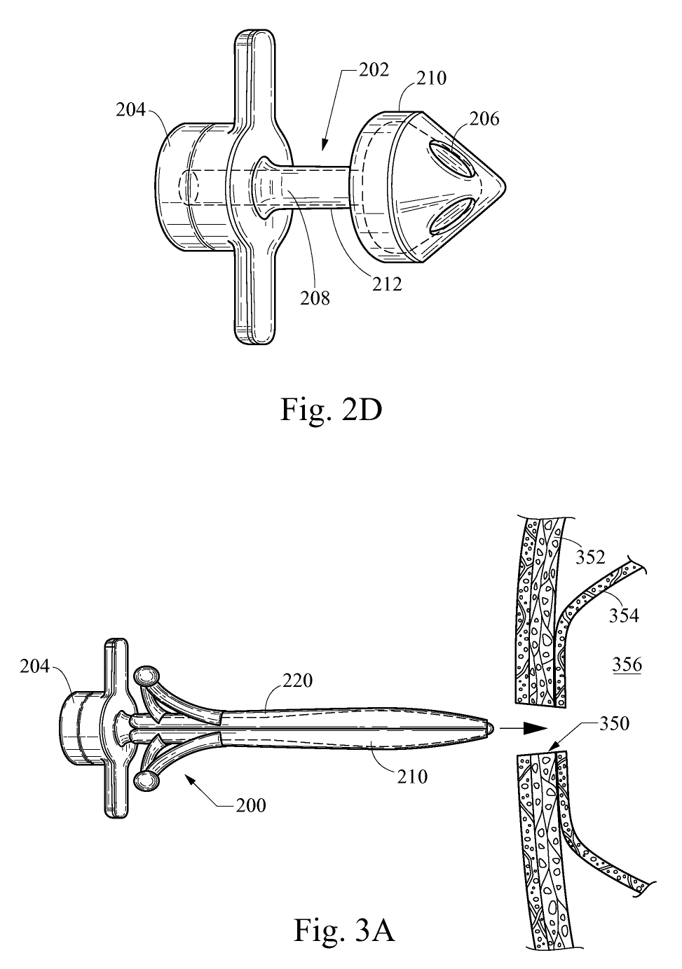

One embodiment of a gastronomy tube device 200 of the present invention is shown with reference to FIGS. 2A-2D. FIG. 2A shows the assembled, undeployed device 200, FIG. 2B shows a disassembled removable sheath configured as a peel-away sheath 220, FIG. 2C shows the elongate generally tubular body 202 of the device 200 in an undeployed configuration (as if constrained by a sheath), and FIG. 2D shows the body 202 in a deployed configuration with the sheath 220 removed.

As shown in FIG. 2A, the assembled, undeployed device 200 includes a peel-away sheath 220 disposed around the distal length of an elongate, generally tubular body 202. FIG. 2C illustrates that the body 202 includes an external proximal bolster portion 204 that has a larger outer diameter than an adjacent length of the undeployed body 202. The distal portion of the body 202 includes at least one distal aperture 206 open to a longitudinal body lumen 208 that provides a path of fluid communication from a proximal end of the bolster 204 to the aperture 206. When the device 200 is in use, the lumen 208 provides for fluid communication into and out of the patient's stomach (e.g., for gastric decompression or drainage, or for delivery of medication or nutrition).

A retention portion, embodied as a distal expandable region 210 of the body 202 is biased into an expanded configuration that is shown in FIG. 2D. This bias preferably is provided by a preconfigured (e.g., molded or otherwise formed) shape of the expandable body region 210 and preferably uses a material configured to be deformable from an original shape by constraint, but that substantially resumes its shape upon removal of the constraint. One example of a desirable material includes polyurethane, but those of skill in the art will appreciate that a number of other polymeric and non-polymeric materials may be used within the scope of the present invention. Such examples include shape-memory materials that may be used to form the expandable portion, or that may be embedded therein. For example the expandable region may include one or more embedded or overmolded strips, wires, or other shaped components including a shape-memory plastic or alloy configured to conform the body to a predetermined expanded shape.

As shown in FIGS. 2A and 2C, the self-expanding portion 210 of the body 202 may be constrained in an undeployed configuration that provides a low profile (i.e., smaller outer diameter than a deployed configuration). This constrained configuration allows a user to direct the distal expandable body region 210 through a stoma of a patient, which stoma is smaller in diameter than both the expanded diameter of that region 210 and of the bolster 204. However, the stoma preferably has a slightly greater inner diameter as compared with the outer diameter of an intermediate body region 212, according to desired medical specifications such that the intermediate body region 212 may rotate freely within the stoma but not have a substantial gap therebetween. Accordingly, the intermediate body region 212 preferably has a substantially static outer diameter that is less than that of the bolster 204 and the expanded outer diameter of the distal expandable region 210. It is preferable that the body 202 be constructed of and/or coated with a biocompatible material, and it is also preferable that the material will provide a lubricious surface and/or be receptive to lubrication without adversely affecting the integrity, biocompatibility, or its other desirable properties.

FIGS. 2A and 2B show a removable sheath configured as a peel-away sheath 220. The sheath 220 includes two sheath portions 222, 224 that are separable from each other. Each sheath portion preferably includes a graspable handle member 226. FIG. 2A shows the sheath 220 intact and constraining the body 202 in its low-profile, undeployed configuration. In order to effect this, it is preferable that the sheath 220 is substantially radially inelastic in a region immediately adjacent the expandable region 210 of the body 202. FIG. 2B shows the sheath 220 having been split into its separate sheath portions 222, 224. Splittable sheath embodiments are known in the art, and one desirable embodiment is disclosed in U.S. Pat. No. 5,320,602 to Karpiel, et al. (and assigned to Wilson-Cook Medical, Inc., Winston-Salem, N.C.), which is incorporated by reference herein in its entirety. Embodiments may be practiced within the scope of the present invention that include one or more of co-extruded sheath portions, sheath portions that are perforated, scored, or otherwise provided with one or more weakened “split zones,” sheath portions that engage each other with a zipper-like or tongue-and-groove-like interface, or any other splittable connection interface along the contacting lateral edges 222a, 224a of the sheath portion 222, 224. The inner-facing and outer-facing surfaces of the sheath portions 222, 224 preferably include lubricious or otherwise low-friction surfaces configured to facilitate minimal frictional engagement with the device body 202 or a patient's stoma during sliding removal of the sheath portions 222, 224 from the device body 202. The sheath alternatively may be configured as having a single splittable region, or more than two splittable regions.

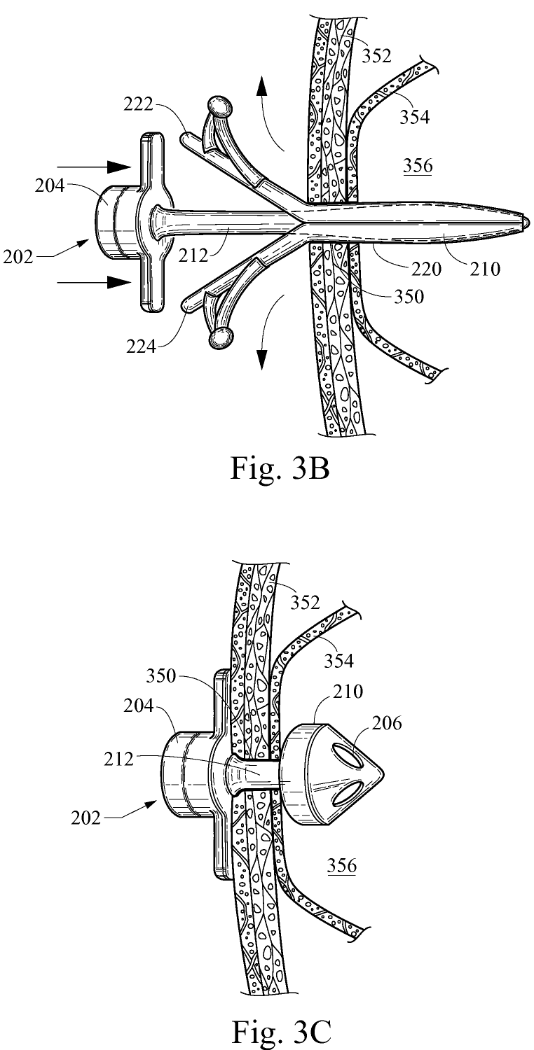

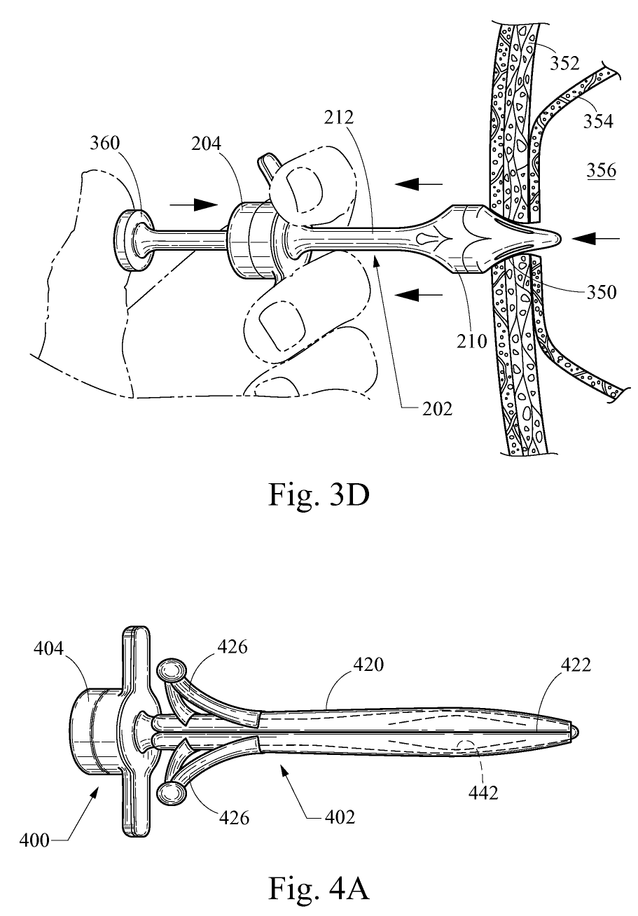

A method of using the gastronomy tube device 200 is described with reference to FIGS. 3A-3D. As shown in FIG. 3A, a gastronomy tube device 200 is provided and is directed adjacent a stoma 350 that extends through the abdominal wall 352 and stomach wall 354 into the stomach cavity 356 of a patient. The exterior surfaces of the sheath 220 may be coated with a medically acceptable lubricant, such as—for example—a water-based lubricant. As shown, in FIG. 3B, the distal portion of the device 200 may be directed through the stoma 350 and the sheath portions 222, 224 withdrawn while being split apart from each other. Removal of the sheath 220 allows the expandable body region 210 of the body 202 to deploy into its default, larger diameter expanded configuration, as is shown in FIG. 3C. In this manner, the expandable body region 210 provides a retaining feature that prevents the device 200 from being passed proximally through the stoma 350. The intermediate region 212 of the body 202 occupies the stoma 350, and the bolster 204 provides a proximal retention feature that prevents the device 200 from passing completely distally through the stoma 350. In a preferred installation, the bolster 204 will be maintained in a manner that does not in contact with, and more preferably is at least a short distance (preferably at least 5 mm) from the skin surface of the patient.

Removal of the body 202 is described with reference to FIG. 3D. To remove the body 202 from a patient stoma 350, an elongate obturator 360 is directed through the body lumen 208. Specifically, a user may press the distal end of the obturator 360 into contact with the distal end of the lumen 208 (not shown, see FIG. 2D) with sufficient force to elongate the body 202 and reduce the outer diameter of the expandable body region 210 sufficiently to ease proximal-ward passage through the stoma 350 (contact with the stoma walls may further reduce the body's outer diameter). Preferably, the user will grippingly engage the proximal bolster 204 to hold it relatively stationary relative to the obturator 360, and will—upon actuation of the obturator 360 to reduce the body's outer diameter—use that gripping engagement to withdraw the body 202 from the patient's stoma 350.

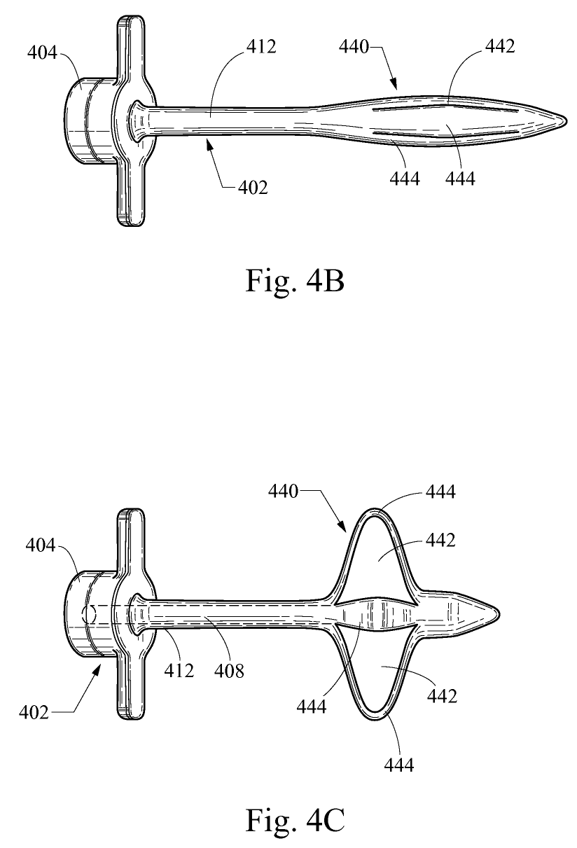

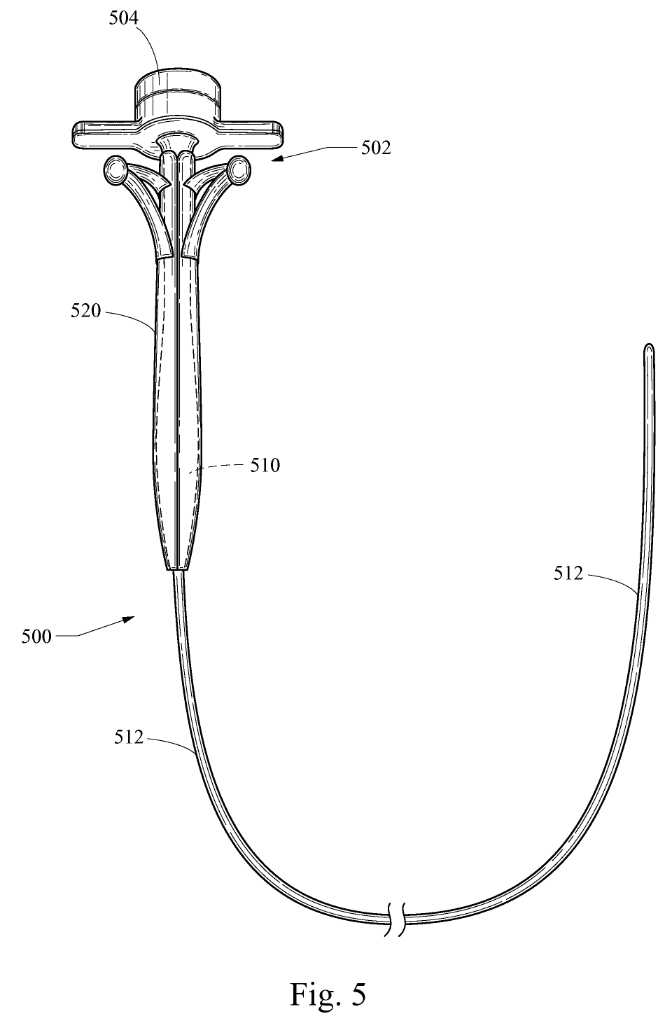

Another embodiment of a gastronomy tube device 400 is described with reference to FIGS. 4A-4C. FIG. 4A shows the assembled, undeployed device 400, FIG. 4B shows the elongate generally tubular body 402 of the device 400 in an undeployed configuration (as if constrained by a sheath), and FIG. 4C shows the body 402 in a deployed configuration with the sheath 420 removed.

As shown in FIG. 4A, the assembled, undeployed device 400 includes a peel-away sheath 420 having a single splittable region 422 disposed around the distal length of an elongate, generally tubular body 402 (shown in dashed outline as constrained by the sheath). FIG. 4B illustrates that the body 402 includes an external proximal bolster portion 404 that has a larger outer diameter than an intermediate lengthwise portion 412 of the undeployed body 402. A self-expanding distal portion of the body 402 is configured as a malecot 440 including slits 442 in the body 402 that define malecot wings 444. The body 402 includes a longitudinal body lumen 408 that provides a path of fluid communication from a proximal end of the bolster 404 to the malecot slits 442 (which form open apertures 442 when the malecot 440 is expanded in the manner depicted in FIG. 4C). When the device 400 is in use, the lumen 408 provides for fluid communication into and out of the patient's stomach (e.g., for gastric decompression or drainage, or for delivery of medication or nutrition).

A distal expandable region of the body 402, configured for use as a retention member, is connected with the bolster 404 by the intermediate body region 412, which has a generally static outer diameter. The distal expandable region of the body 402, conformed as the malecot 440, is biased into an expanded configuration that is shown in FIG. 4C. This bias preferably is provided by a treating the malecot wings (e.g., by heat, chemical fixative, or mechanical pressure) to assume—when not constrained—the expanded conformation illustrated in FIG. 4C, and/or by using a material configured to be deformable from an original shape by constraint, but that substantially resumes that original shape upon removal of the constraint. One example of a desirable material includes polyurethane, but those of skill in the art will appreciate that a number of other polymeric and non-polymeric materials may be used within the scope of the present invention. For example, one or more of the malecot wings may incorporate a shape-memory material configured to, upon release of the sheath's circumferential constraint, direct the malecot wing to assume a predetermined expanded configuration.

As shown in FIGS. 4A and 4B, the body 402 may be constrained in an undeployed configuration that provides a low profile (i.e., smaller outer diameter than a deployed configuration). This constrained configuration allows a user to direct the distal expandable body region 410 through a stoma of a patient, which stoma is smaller in diameter than both the expanded diameter of that region 410 and of the bolster 404. However, the stoma preferably has a slightly greater inner diameter as compared with the outer diameter of an intermediate body region 412, according to desired medical specifications such that the intermediate body region 412 may rotate freely within the stoma but not have a substantial gap therebetween. Accordingly, it is preferable that the body 402 be constructed of a biocompatible material, and it is also preferable that the material will provide a lubricious surface and/or be receptive to lubrication without adversely affecting the integrity, biocompatibility, or its other desirable properties.

FIG. 4A shows a removable sheath configured as a peel-away sheath 420. The sheath 420 includes a single splittable region 422 and preferably includes at least one graspable handle member 426 configured to facilitate pulling the sheath 420 proximally relative to the body 402 and actuating the splittable region 422 (e.g., by pulling it open/apart). FIG. 4A shows the sheath 420 intact and constraining the body 402 in its low-profile, undeployed configuration. In order to effect this, it is preferable that the sheath 420 is substantially radially inelastic in a region immediately adjacent the malecot portion 440 of the body 402. A method of use for the embodiment shown in FIGS. 4A-4B may be implemented in a manner substantially similar to that described above with reference to FIGS. 3A-3D. Alternatively, for this embodiment or a variant thereof being practiced within the scope of the present invention, the malecot may be deployed using another deployment means as known in the art such as, for example, a deployment sheath that is incorporated into the body of the device.

Another embodiment of a gastronomy tube device 500 of the present invention is shown with reference to FIG. 5. FIG. 5 shows the assembled, undeployed device 500 with a removable sheath configured as a peel-away sheath 520. 'This embodiment is configured with a longer distal tube portion 512 that may extend into a patient's small intestine (e.g., the duodenum, jejunum). The deployable/expandable portion 510 is configured in the same manner as the other embodiments, except that it may be configured without any apertures such that a path of fluid communication is provide to (or near) the distal end of the tube. The body 202 includes an external proximal bolster portion 204 that has a larger outer diameter than an adjacent length of the undeployed body 202. As shown in this embodiment, different proportions of device components may be used within the scope of the present invention, including that the terms “gastronomy tube” or “g-tube” should be understood to encompass similar devices that provide fluid communication to other body sites including, for example, a “jejunostomy tube” (“j-tube”).

Many modifications and other aspects of the invention may come to mind to one skilled in the art to which this invention pertains upon having the benefit of the teachings presented in the foregoing description; and it will be apparent to those skilled in the art that variations and modifications of the present invention can be made without departing from the scope or spirit of the invention. Therefore, it is to be understood that the invention is not to be limited to the specific aspects disclosed and that modifications and other aspects are intended to be included within the scope of the claims, which define the metes and bounds of the present invention. Although specific terms are employed herein, they are used in a generic and descriptive sense only and not for purposes of limitation.

1. A medical device comprising:
a gastronomy tube configured as an elongate generally tubular body including a proximal end and a distal end with an elongate lumen disposed through its length, where the body includes a distal expandable region that is biased toward an expanded configuration; and
a removable sheath disposed around and substantially constraining the expandable region in a non-expanded configuration having an outer diameter less than the expanded configuration.

2. The medical device of claim 1, where the expandable region comprises a malecot including at least one malecot wing.

3. The medical device of claim 1, where the removable sheath is substantially radially inelastic in a region immediately adjacent the expandable region of the body.

4. The medical device of claim 1, further comprising:
an obturator configured for elongating the body in a manner reducing the outer diameter of the expanded configuration.

5. The medical device of claim 1, where the removable sheath comprises a peel-away sheath having at least one splittable region.

6. The medical device of claim 1, where a removal of the removable sheath from the body allows the expandable region to assume its biased expanded configuration.

7. The medical device of claim 1, where the proximal end comprises a bolster portion configured to retain the proximal portion externally relative to a patient stoma.

8. The medical device of claim 7, where the body comprises an intermediate region having a substantially static outer diameter that is less than an outer diameter of the bolster portion and is less than an outer diameter of the expanded configuration of the expandable region.

9. A medical device comprising:
a gastronomy tube device configured as an elongate generally tubular body including a proximal end and a distal end with an elongate lumen disposed through substantially its entire length, where the body includes a distal self-expanding portion; and
a removable sheath disposed around and substantially constraining the distal self-expanding portion in a non-expanded configuration having an outer diameter that is less than the expanded configuration.

10. The medical device of claim 9, where the removable sheath comprises at least one splittable region.

11. The medical device of claim 9, where the removable sheath is substantially radially inelastic in a region immediately adjacent the self-expanding portion of the body.

12. The medical device of claim 9, where the distal self-expanding portion is molded in an expanded configuration that is circumferentially constrained by the sheath.

13. The medical device of claim 9, where the distal self-expanding portion comprises a shape-memory material.

14. The medical device of claim 9, where a removal of the removable sheath from the body allows the distal self-expanding portion to assume its expanded configuration.

15. The medical device of claim 9, where the distal self-expanding portion comprises a malecot including at least one malecot wing.

16. The medical device of claim 9, where the proximal end comprises a bolster portion separated from the distal self-expanding portion by an intermediate portion, the intermediate region having a substantially static outer diameter that is less than an outer diameter of the bolster portion and is less than an outer diameter of the expanded configuration of the distal self-expanding portion.

17. A method of using a gastronomy tube device comprising the steps of:
providing a gastronomy tube device comprising a self-expanding distal retention member that is constrained by a removable sheath;
directing the distal retention member to a target site; and
removing the sheath, allowing expansion of an outer diameter of the distal self-expanding retention member.

18. The method of claim 17, where a proximal end of the gastronomy tube device further comprises a bolster portion separated from the distal self-expanding retention member by an intermediate portion, the intermediate region having a substantially static outer diameter that is less than an outer diameter of the bolster portion and is less than an expanded outer diameter of the distal self-expanding retention member.

19. The method of claim 17, further comprising the steps of:
providing an elongate obturator;
directing a distal end of the obturator through a length of the device in manner that elongates and reduces the outer diameter of the distal self-expanding retention member; and
withdrawing the device from the target site.

20. The method of claim 17, where the target site comprises a stoma providing access through a patient abdominal wall to a patient stomach cavity.
